(19) United States
(12) Patent Application Publication
(10) Pub. No.: US 2025/0256019 A1
(43) Pub. Date: Aug. 14, 2025

(54) NASAL ASPIRATOR

(71) Applicant: SHENZHEN ZXD TRADING CO., LTD., Shenzhen (CN)

(72) Inventor: Wei Yi, Shenzhen (CN)

(21) Appl. No.: 18/611,977

(22) Filed: Mar. 21, 2024

(30) Foreign Application Priority Data
Feb. 10, 2024 (CN) 202410179108.5

(51) Int. Cl. A61M 1/00

(52) U.S. Cl. CPC A61M 1/682; A61M 2205/50

(57) ABSTRACT

A nasal aspirator includes a housing assembly, a negative pressure assembly and a guide assembly. A negative pressure cavity and a treatment cavity are arranged in the housing assembly at intervals, the treatment cavity is sequentially divided into a first cavity chamber and a second cavity chamber by a partition plate, and the first cavity chamber is communicated with the second cavity chamber; the negative pressure assembly is arranged in the negative pressure cavity and is formed with a suction pipeline and a discharge pipeline which are communicated with a side wall surface of the housing assembly; the guide assembly comprises a first guide piece and a second guide piece, and the first guide piece is detachably connected with the side wall surface of the housing assembly; and the second guide piece is fixedly connected to the side wall surface of the housing assembly.

### CROSS REFERENCE TO RELATED APPLICATIONS

The present application claims the benefit of Chinese Patent Application No. 202410179108.5 filed on Feb. 10, 2024, the contents of which are incorporated herein by reference in their entirety.

### TECHNICAL FIELD

The present application belongs to the technical field of medical appliances, and more particularly, relates to a nasal aspirator.

### BACKGROUND

In the process of pediatric diagnosis and treatment, because children's body structures are still fragile, it is necessary to be extra cautious in medical care for children, especially in common situations such as nasal allergies or colds in children, solid, liquid or mixed solid-liquid secretions often appear in nasal cavities of children. Therefore, it is necessary to clean the secretions in time without damaging children's nasal body structures.

In the related art, nasal cavity cleaners or nasal cavity aspirators are usually used to treat nasal cavity secretions. The former drips liquid into the nasal cavity to moisten the secretions, and then squeeze and wash out the secretions by water flow. The latter draws the secretions out and treats the secretions by air in the form of negative pressure. Because the water flow in the working process of the nasal cavity cleaners may drip out or overflow and pollute the environment, the nasal cavity aspirators in the form of negative pressure are gradually widely used due to the features of simple working process and easy cleaning thereof.

In the nasal aspirators in the form of negative pressure, the two most widely used are hand-held nasal aspirators and bench-style nasal aspirators. However, the former is limited by a size demand, and an internal negative pressure assembly usually cannot provide suction of large power; the latter can provide enough power, but a trachea used to guide secretion suction is difficult to disassemble and clean, and a mounting and maintenance process is complicated.

### SUMMARY

Embodiments of the present application are intended to provide a nasal aspirator, which aims at improving a problem that a current nasal aspirator is not convenient to clean.

In order to achieve the above object, the present application provides a nasal aspirator, comprising a housing assembly, a negative pressure assembly and a guide assembly; a negative pressure cavity and a treatment cavity are arranged in the housing assembly at intervals, the treatment cavity is sequentially divided into a first cavity chamber and a second cavity chamber by a partition plate, and the first cavity chamber is communicated with the second cavity chamber; the negative pressure assembly is arranged in the negative pressure cavity and is formed with a suction pipeline and a discharge pipeline which are communicated with a side wall surface of the housing assembly; the guide assembly comprises a first guide piece and a second guide piece, and the first guide piece is detachably connected with the side wall surface of the housing assembly to be communicated with the suction pipeline and the second cavity chamber; and the second guide piece is fixedly connected to the side wall surface of the housing assembly and is communicated with the discharge pipeline and the first cavity chamber.

As a feasible embodiment, the first guide piece comprises a first guide tube and a soft rubber dropper, the first guide tube is made of a flexible material and arranged at the side wall surface of the housing assembly to be communicated with the suction pipeline; the soft rubber dropper is made of a flexible material and fixedly connected to one end of the first guide tube away from the housing assembly, one end of the soft rubber dropper away from the first guide tube is provided with a liquid suction port, and the liquid suction port is detachably connected to the side wall surface of the housing assembly to be communicated with the first guide tube and the second cavity chamber.

As a feasible embodiment, the first guide piece further comprises a first joint and a second joint, the first joint is arranged on one side wall surface of the housing assembly close to the negative pressure cavity and is communicated with the suction pipeline, and the second joint is arranged on one side wall surface of the housing assembly close to the second cavity chamber and is communicated with the second cavity chamber; one side of the first guide tube away from the soft rubber dropper is communicated with the first joint; and the soft rubber dropper is detachably connected to the second joint to enable the liquid suction port to be communicated with the second joint.

As a feasible embodiment, the first guide tube and the soft rubber dropper are integrally formed. As a feasible embodiment, the second guide piece comprises a third joint, a fourth joint and a second guide tube, and the third joint is arranged on one side wall surface of the housing assembly close to the negative pressure cavity and is communicated with the discharge pipeline; the fourth joint is arranged on one side wall surface of the housing assembly close to the first cavity chamber and is communicated with the first cavity chamber; and the second guide tube is made of a flexible material and is provided with two opposite ends to be communicated with the third joint and the fourth joint respectively.

As a feasible embodiment, the housing assembly comprised a base and a water tank, the base is formed with the negative pressure cavity and a mounting groove, and the negative pressure cavity and the mounting groove are arranged at intervals; and the water tank is detachably connected to the mounting groove and enclosed with an inner wall surface of the mounting groove to form the treatment cavity, and the partition plate is arranged at the water tank.

As a feasible embodiment, the base comprises a base body and a cover plate, and the mounting groove is arranged at the base body; and the cover plate is detachably connected to the base body and enclosed with a wall surface of the base body to form the negative pressure cavity.

As a feasible embodiment, a side wall surface of the water tank is convexly provided with at least two clamping protrusions, the at least two clamping protrusions are symmetrically arranged along a detachable connection direction of the water tank, the inner wall surface of the mounting groove is concavely provided with at least two clamping grooves, and a number of the clamping grooves is set corresponding to the clamping protrusions to clamp the clamping protrusions when the water tank is detachably connected in the mounting groove.

As a feasible embodiment, the housing assembly further comprises a filter piece, and the filter piece is arranged at the partition plate and is communicated with the first cavity chamber and the second cavity chamber.

As a feasible embodiment, the nasal aspirator further comprises a control assembly, the control assembly is electrically connected with the negative pressure assembly, an inner wall surface of the negative pressure cavity is convexly provided with an accommodating step, the mounting groove is arranged on one side of the accommodating step away from the negative pressure cavity, and the control assembly is arranged at the accommodating step.

Compared with the prior art, the nasal aspirator provided by the embodiments of the present application has the beneficial effects as follows:
the negative pressure cavity and the treatment cavity arranged at intervals are formed through the housing assembly, and the treatment cavity is divided into the first cavity chamber and the second cavity chamber by the partition plate; the negative pressure assembly arranged in the negative pressure cavity provides suction from the suction pipeline to the discharge pipeline; the first guide piece in the guide assembly sucks nasal secretions of a user and then the secretions are guided by the suction provided by the negative pressure assembly to sequentially pass through the first guide piece, the suction pipeline, the discharge pipeline and the second guide piece to enter the first cavity chamber and move towards the second cavity chamber; after use, the first guide piece is detachably communicated with the second cavity chamber and is influenced by a fluid in the second cavity chamber to realize a self-cleaning function, thus further improving convenience of cleaning and maintenance as well as mounting and production efficiency of the nasal aspirator.

### BRIEF DESCRIPTION OF THE DRAWINGS

In order to more clearly illustrate the technical solutions of the embodiments of the present application, the drawings that are required to describe the embodiments or the prior art will be briefly introduced below. Apparently, the drawings that are described below are only some embodiments of the present application, and those of ordinary skills in the art can obtain other drawings according to these drawings without going through any creative work.

FIG. 1 is a first schematic three-dimensional structure diagram of a nasal aspirator provided by an embodiment of the present application;

FIG. 2 is a schematic explosion structure diagram of the nasal aspirator provided by the embodiment of the present application;

FIG. 3 is a horizontal schematic cross-sectional structural diagram of the nasal aspirator provided by the embodiment of the present application;

FIG. 4 is a longitudinal schematic cross-sectional structural diagram of a housing assembly adopted in the embodiment of the present application;

FIG. 5 is a second schematic three-dimensional structure diagram of a water tank adopted in the embodiment of the present application;

FIG. 6 is a schematic cross-sectional view of the water tank according to the embodiment of the present application; and

FIG. 7 is an enlarged view of a structure at a part A in FIG. 6.

### REFERENCE NUMERALS IN THE DRAWINGS ARE AS FOLLOWS

100 refers to nasal aspirator; 10 refers to housing assembly; 11 refers to base; 111 refers to negative pressure cavity; 112 refers to accommodating step; 113 refers to mounting groove; 115 refers to base body; 116 refers to reinforcing rib; 117 refers to cover plate; 13 refers to water tank; 131 refers to treatment cavity; 133 refers to first cavity chamber; 135 refers to second cavity chamber; 137 refers to clamping protrusion; 15 refers to partition plate; 17 refers to filter piece; 18 refers to elastic piece; 30 refers to negative pressure assembly; 31 refers to suction pipeline; 33 refers to discharge pipeline; 50 refers to guide assembly; 51 refers to first guide piece; 511 refers to first guide tube; 513 refers to soft rubber dropper; 515 refers to first joint; 517 refers to second joint; 53 refers to second guide piece; 531 refers to third joint; 533 refers to fourth joint; 535 refers to second guide tube; and 70 refers to control assembly.

### DETAILED DESCRIPTION

To make the technical problems to be solved, technical solutions, and beneficial effects of the present application clearer, the present application will be further explained in detail with reference to the drawings and embodiments. It should be understood that the specific embodiments described herein are merely illustrative of the present application and are not intended to limit the present application.

It should be noted that all directional indicators (such as up, down, left, right, front, back, and the like) in the embodiments, are only used to explain the relative positional relationships and movement situations between components in a certain posture (as shown in the attached drawings). If the specific posture changes, the directional indicators will also change accordingly.

In the present invention, unless otherwise specified and limited, terms such as “connection” and “fixation”, etc., should be understood broadly, for example, the “connection” may be fixed connection, or detachable connection or integral connection; may be mechanical connection, and may also be electrical connection; and may be direct connection, may also be indirect connection through an intermediate medium, and may also be internal communication of two elements or interaction relationship of two elements, unless otherwise explicitly defined. The specific meaning of the above terms in the present invention can be understood in a specific case by those of ordinary skills in the art.

In addition, the descriptions related to “first”, “second”, etc. in the present invention are used for descriptive purposes only and cannot be understood as indicating or implying relative importance, or implicitly indicating the number of technical features indicated thereby. Therefore, the features defined with “first” and “second” can explicitly or implicitly comprise at least one of the features. In addition, “and/or” appearing in the whole text means three parallel solutions, for example, “A and/or B” means the solution that A satisfies, the solution that B satisfies or the solution that A and B satisfy at the same time. In addition, the technical solutions of various embodiments may be combined with each other, but have to be based on what those having ordinary skills in the art can achieve. When the combination of the technical solutions is contradictory or impossible to realize, it should be considered that the combination of such technical solutions does not exist and is not within the scope of protection required by the present invention.

In the process of pediatric diagnosis and treatment, because children's body structures are still fragile, it is necessary to be extra cautious in medical care for children, especially in common situations such as nasal allergies or colds in children, solid, liquid or mixed solid-liquid secretions often appear in nasal cavities of children. Therefore, it is necessary to clean the secretions in time without damaging children's nasal body structures.

In the related art, nasal cavity cleaners or nasal cavity aspirators are usually used to treat nasal cavity secretions. The former drips liquid into the nasal cavity to moisten the secretions, and then squeeze and wash out the secretions by water flow. The latter draws the secretions out and treats the secretions by air in the form of negative pressure. Because the water flow in the working process of the nasal cavity cleaners may drip out or overflow and pollute the environment, the nasal cavity aspirators in the form of negative pressure are gradually widely used due to the features of simple working process and easy cleaning thereof.

In the nasal aspirators in the form of negative pressure, the two most widely used are hand-held nasal aspirators and bench-style nasal aspirators. However, the former is limited by a size demand, and an internal negative pressure assembly usually cannot provide suction of large power; the latter can provide enough power, but a trachea used to guide secretion suction is difficult to disassemble and clean, and a mounting and maintenance process is complicated.

The embodiments of the present application are intended to provide a nasal aspirator, which aims at improving the defects of the prior art, further improving convenience of cleaning and maintenance and reducing a production cost of the nasal aspirator on the premise of ensuring sufficient suction.

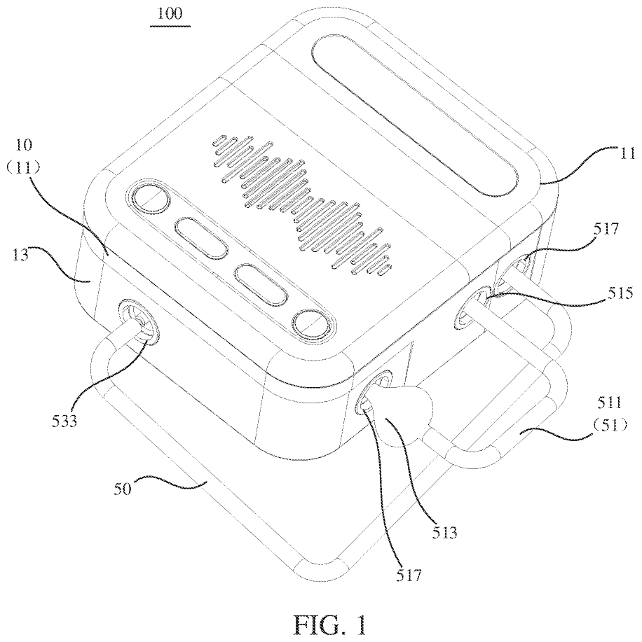

With reference to FIG. 1 to FIG. 5, a nasal aspirator 100 provided by an embodiment of the present application will be described in detail hereinafter through specific embodiments.

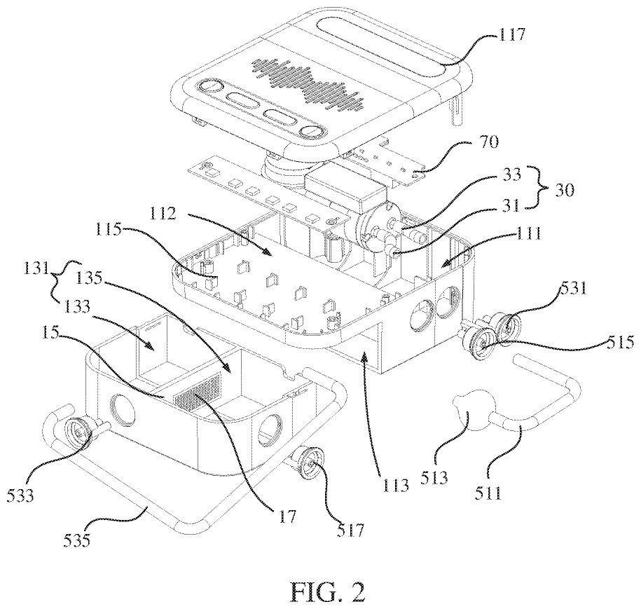

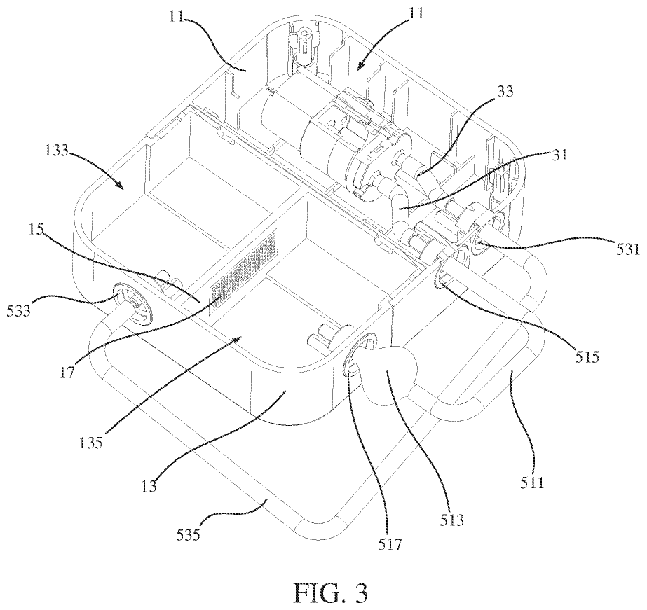

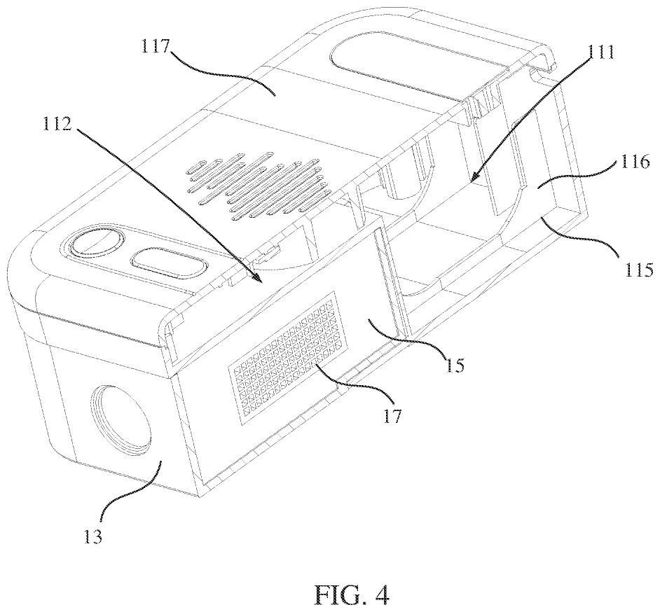

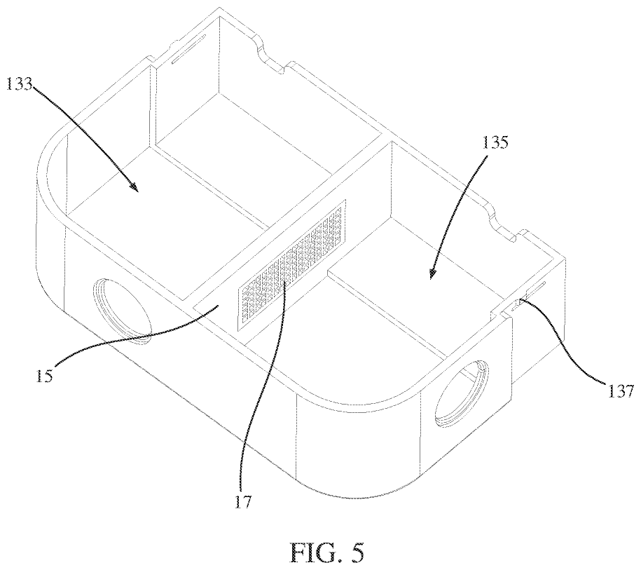

Please refer to FIG. 1 to FIG. 5 together, FIG. 1 is a first schematic three-dimensional structure diagram of the nasal aspirator 100 provided by the embodiment of the present application; FIG. 2 is a schematic explosion structure diagram of the nasal aspirator 100 provided by the embodiment of the present application; FIG. 3 is a horizontal schematic cross-sectional structural diagram of the nasal aspirator 100 provided by the embodiment of the present application; FIG. 4 is a longitudinal schematic cross-sectional structural diagram of a housing assembly 10 adopted in the embodiment of the present application; and FIG. 5 is a second schematic three-dimensional structure diagram of a water tank 13 adopted in the embodiment of the present application.

One embodiment of the present invention provides a nasal aspirator 100, comprising a housing assembly 10, a negative pressure assembly 30 and a guide assembly 50. A negative pressure cavity 111 and a treatment cavity 131 are arranged in the housing assembly 10 at intervals, the treatment cavity 131 is sequentially divided into a first cavity chamber 133 and a second cavity chamber 135 by a partition plate 15, and the first cavity chamber 133 is communicated with the second cavity chamber 135. The negative pressure assembly 30 is arranged in the negative pressure cavity 111 and is formed with a suction pipeline 31 and a discharge pipeline 33 which are communicated with a side wall surface of the base 11. The guide assembly 50 comprises a first guide piece 51 and a second guide piece 53, the first guide piece 51 is detachably connected with the side wall surface of the housing assembly 10 to be communicated with the suction pipeline 31 and the second cavity chamber 135. The second guide piece 53 is fixedly connected to the side wall surface of the housing assembly 10 and is communicated with the discharge pipeline 33 and the first cavity chamber 133.

It can be understood that the housing assembly 10 is used to bear the negative pressure assembly 30, the guide assembly 50 and other possible assemblies in the nasal aspirator 100, and provide a relatively fixed positional relationship for each assembly, thus ensuring stable realization of a working process of the nasal aspirator 100. Specifically, the negative pressure cavity 111 is arranged in the housing assembly 10 to accommodate the negative pressure assembly 30, and the partition plate 15 is arranged to divide another treatment cavity 131 into the first cavity chamber 133 and the second cavity chamber 135, wherein the first cavity chamber 133 is used for receiving sucked nasal secretions of a user, and the second cavity chamber 135 is used for receiving unpolluted or filtered clarified water. The clarified water may be clean water, salt solution or cleaning liquid with the above components, which is not specifically limited in the present application.

It should be noted that the negative pressure assembly 30 may be a rotary-vane suction pump, a piston suction pump, a liquid ring suction pump or a diaphragm suction pump, and the like, which is not specifically limited in the present application, as long as the negative pressure assembly can guide the secretions in the nasal cavity of the user to be sucked into the suction pipeline 31 through the first guide piece 51 and discharged from the discharge pipeline 33 toward the first cavity chamber 133 through the second guide piece 53. The treatment cavity 131 and the negative pressure cavity 111 arranged at intervals make environments in the two cavities independent from each other, which can avoid possible influence of the negative pressure environment of the negative pressure cavity 111 on the treatment cavity 131, and further improve working stability of the nasal aspirator 100.

It can be understood that one end of the first guide piece 51 in the guide assembly 50 away from the suction pipe 31, is used to extend into the nasal cavity of the user and suck out the secretions in the nasal cavity of the user and guide the secretions into the suction pipeline 31 while providing suction toward the nasal cavity of the user. The negative pressure assembly 30 guides the secretions to be discharged through the discharge pipeline 33 and enter the first cavity chamber 133 under the guidance of the second guide piece 53. The secretions in the first cavity chamber 133 are diluted or cleaned by the clean water, salt solution or cleaning solution with other components carried in the first cavity chamber, and a clean water body without secretion flows to the second cavity chamber 135. After use, the first guide piece 51 is used to movably splice and communicate with the second cavity chamber 135 with one end extending into the nasal cavity of the user. In this case, the negative pressure assembly 30 provides clean water flow power for the cleaning process of the first guide piece 51, and the nasal aspirator 100 realizes the “self-cleaning” effect on the secretions, further improving convenience of use of the nasal aspirator 100.

It should be noted that the first guide piece 51 and the second guide piece 53 are provided separately. When the first guide piece 51 and/or the second guide piece 53 need to be maintained or replaced due to the loss in the working process, only the first guide piece 51 and/or the second guide piece 53 that has suffered losses need to be replaced correspondingly, and other parts such as the housing assembly 10 or the negative pressure assembly 30 are not required to be replaced, which further improves the mounting and maintenance efficiency of the nasal aspirator 100 and reduces the production cost.

As a feasible embodiment, the first guide piece 51 comprises a first guide tube 511 and a soft rubber dropper 513, the first guide tube 511 is made of a flexible material and arranged at the side wall surface of the housing assembly 10 to be communicated with the suction pipeline 31. The soft rubber dropper 513 is made of a flexible material and fixedly connected to one end of the first guide tube 511 away from the housing assembly 10, one end of the soft rubber dropper 513 away from the first guide tube 511 is provided with a liquid suction port, and the liquid suction port is detachably connected to the side wall surface of the housing assembly 10 to be communicated with the first guide tube 511 and the second cavity chamber 135.

As a feasible embodiment, the first guide piece 51 further comprises a first joint 515 and a second joint 517. The first joint 515 is arranged on one side wall surface of the housing assembly 10 close to the negative pressure cavity 111 and is communicated with the suction pipeline 31. The second joint 517 is arranged on one side wall surface of the housing assembly 10 close to the second cavity chamber 135 and is communicated with the second cavity chamber 135. One side of the first guide tube 511 away from the soft rubber dropper 513 is communicated with the first joint 515. The soft rubber dropper 513 is detachably connected to the second joint 517 to enable the liquid suction port to be communicated with the second joint 517.

It can be understood that the soft rubber dropper 513 is made of flexible material, and a shape thereof is preferably set corresponding to a nasal cavity shape of a user, so as to better fit the nasal cavity shape of the user without affecting the secretion suction process, avoid possible damage to the nasal cavity of the user, and further improve use comfort of the nasal aspirator 100. Specifically, the material of the soft rubber dropper 513 may be medical silica gel, rubber, polyvinyl chloride or thermoplastic elastomer, and the like, which is not specifically limited in the present application. When the first guide piece 51 is communicated with the treatment cavity 131, the first guide piece may at least partially penetrate the side wall surface of the housing assembly 10, or may be guided through the first joint 515 and the second joint 517, which are respectively communicated with each other on a wall surface of the water tank 13. Compared with the former, the latter further simplifies a mounting process of the first guide tube 511 in the first guide piece 51 through the first joint 515 and the second joint 517 pre-arranged on the side wall surface of the water tank 13, and at the same time, ensures a sealing performance of the first guide piece 51 to the guided secretions during the working process of the nasal aspirator 100, avoiding interference possibly caused by environmental factors such as external dust or liquid drops, and further improving working stability and flexibility of the nasal aspirator 100.

As a feasible embodiment, the first guide tube 511 and the soft rubber dropper 513 are integrally formed.

It can be understood that the material of the first guide tube 511 made of the flexible material may be medical silica gel, rubber, polyvinyl chloride or thermoplastic elastomer, and the like, which is not specifically limited in the present application. Further, the first guide tube 511 and the second guide tube 535 are made of the same material and are integrally formed, that is, the production process of the first guide tube 511 and the soft rubber dropper 513 is realized in a single machining process, which further reduces a production procedure of the first guide tube 511 and the soft rubber dropper 513 in the nasal aspirator 100, that is, reduces a production cycle of the nasal aspirator 100 and improves the production efficiency. On the other hand, the first guide tube 511 and the soft rubber dropper 513, which are integrally formed, form a whole, which does not have a possibility of leakage at the joint, further improves the sealing performance of the first guide tube 511 and the soft rubber dropper 513, and also reduces occurrence of connection and seaming, so that an appearance of the nasal aspirator 100 is more beautiful and can better fit the nasal cavity shape of the user, that is, the working stability and use comfort of the nasal aspirator 100 are further increased.

As a feasible embodiment, the second guide piece 53 comprises a third joint 531, a fourth joint 533 and a second guide tube 535. The third joint 531 is arranged on one side wall surface of the housing assembly 10 close to the negative pressure cavity 111 and is communicated with the discharge pipeline 33. The fourth joint 533 is arranged on one side wall surface of the housing assembly 10 close to the first cavity chamber 133 and is communicated with the first cavity chamber 133. The second guide tube 535 is made of a flexible material and is provided with two opposite ends to be communicated with the third joint 531 and the fourth joint 533 respectively.

It can be understood that the third joint 531 and the fourth joint 533 arranged on the side wall surface of the housing assembly 10 are used for the communication between the second guide tube 535 in the second guide piece 53 and the first cavity chamber 133 formed in the negative pressure cavity 111. Compared with the arrangement that the second guide piece 53 directly penetrates through the side wall surface of the housing assembly 10 and at least partially extends into the first cavity chamber 133, this arrangement not only ensures a communication effect between the second guide tube 535 and the first cavity chamber 133, but also further simplifies the mounting process of the second guide piece 53 and the nasal aspirator 100. The communication effect can be achieved by means of sleeved connection, clamping connection or splicing between the third joint 531 and the fourth joint and the second guide piece 53, without needing additional structures or tools for mounting, which further improves the production efficiency and the convenience of mounting and maintenance of the nasal aspirator 100.

As a feasible embodiment, the housing assembly 10 comprises a base 11 and a water tank 13, the base 11 is formed with the negative pressure cavity 111 and a mounting groove 113, and the negative pressure cavity 111 and the mounting groove 113 are arranged at intervals. The water tank 13 is detachably connected to the mounting groove 113 and enclosed with an inner wall surface of the mounting groove 113 to form the treatment cavity 131, and the partition plate 15 is arranged on the water tank 13.

It can be understood that the base 11 in the housing assembly 10 is provided with the negative pressure cavity 111 for accommodating the negative pressure assembly 30, and the mounting groove 113 is provided for the process of detachably connecting the water tank 13 to the base 11, so that the water tank 13 can at least partially extend into the mounting groove 113 and be fixed relative to the mounting groove 113 by means of clamping or splicing. During the working process of the nasal aspirator 100, when the clean water, saline solution or cleaning liquid with other components is added to the first cavity chamber 133 and/or the second cavity chamber 135 in the water tank 13, or when the water tank 13 needs to be maintained or replaced due to loss in the working process, the water tank 13 only needs to be detached and taken out separately for relevant treatment, thus avoiding the influence on other components such as the base 11 or the negative pressure assembly 30, and further improving the working convenience as well as the mounting and maintenance efficiency of the nasal aspirator 100.

As a feasible embodiment, the base 11 comprises a base body 115 and a cover plate 117, and the mounting groove 113 is arranged at the base body 115. The cover plate 117 is detachably connected to the base body 115 and enclosed with a wall surface of the base body 115 to form the negative pressure cavity 111.

It can be understood that the base body 115 and the cover plate 117 are detachably connected and enclosed to form the negative pressure cavity 111, so that at least part of the negative pressure cavity 111 can be exposed to the outside by disassembling the cover plate 117, which further facilitates the process of mounting the negative pressure assembly 30 in the negative pressure cavity 111. At the same time, the cover plate 117 and the base body 115 may be made of different materials to adapt to different needs and application scenarios. For example, the base body 115 is made of a transparent material, so that concrete conditions of the water tank 13 in the mounting groove 113 and the negative pressure assembly 30 in the negative pressure cavity 111 can be directly observed by naked eyes, which further improves working flexibility and convenience of mounting and maintenance of the housing assembly 10 and the nasal aspirator 100.

It should be noted that the seat body 115 or the cover plate 117 with different sizes may be replaced according to different needs and application scenarios, so as to change a volume of the negative pressure cavity 111 and then match the negative pressure assemblies 30 with different sizes, shapes and powers, thus greatly improving the working flexibility of the nasal aspirator 100. On the other hand, the seat body 115 and the cover plate 117 with the same specifications may be stacked, stored or transported separately before the assembly process, which further reduces occupied space and provides convenience for assembly and transportation process, and further reduces the production cost as well as mounting and maintenance cost of the nasal aspirator 100.

As a feasible embodiment, as shown in FIG. 4, the housing assembly 10 further comprises a reinforcing rib 116. The reinforcing rib 116 is arranged on one side of the seat body 115 close to the negative pressure cavity 111. The reinforcing rib 116 may be a sheet-like structure with a width direction thereof perpendicular to a surface of the seat body 115 close to the negative pressure cavity 111. The reinforcing rib 116 and the seat body 115 may be integrally connected by injection molding or welded, which can make the seat body 115 difficult to deform under the action of an external force, and make the structure of the seat body 115 firmer and more stable.

As a feasible embodiment, a side wall surface of the water tank 13 is convexly provided with at least two clamping protrusions 137, and the at least two clamping protrusions 137 are symmetrically arranged along a detachable connection direction of the water tank 13. The inner wall surface of the mounting groove 113 is concavely provided with at least two clamping grooves, and a number of the clamping grooves is set corresponding to the clamping protrusions 137 to clamp the clamping protrusions 137 when the water tank 13 is detachably connected in the mounting groove 113.

It can be understood that the water tank 13 detachably connected in the mounting groove 113 tends to be separated from the mounting groove 113 along a notch direction of the mounting groove 113. Thus, the inner wall surface of the mounting groove 113 is provided with the clamping grooves, and the corresponding side wall surface of the water tank 13 is convexly provided with the clamping protrusions 137. The clamping protrusions 137 clamped in the clamping groove prevent the water tank 13 from being separated from the mounting groove 113, so that the water tank 13 mounted in the mounting groove 113 has a relatively fixed positional relationship with the base 11, that is, the working stability of the housing assembly 10 and the nasal aspirator 100 is further improved.

As a feasible embodiment, the housing assembly 10 further comprises a filter piece 17, and the filter piece 17 is arranged at the partition plate 15 and is communicated with the first cavity chamber 133 and the second cavity chamber 135.

It can be understood that the filter piece 17 is used to prevent the secretions in the first cavity chamber 133 from entering the second cavity chamber 135 and polluting the clean water in the second cavity chamber 135. Specifically, a filtering means of the filter piece may be mechanical filtering, activated carbon filtering, nanofiltration membrane, ion exchange, and the like, which is not specifically limited in the present application. In a preferred embodiment, the filter piece 17 of the present application is arranged as a strainer and fixed in a middle of the partition plate 15, so that in the process of liquid flowing from the first cavity chamber 133 to the second cavity chamber 135 through the strainer, the secretions will be blocked at one side of the strainer close to the first cavity chamber 133, so that the water used to clean the first guide piece 51 in the second cavity chamber 135 is always kept clean, further ensuring the self-cleaning effect and working stability of the nasal aspirator.

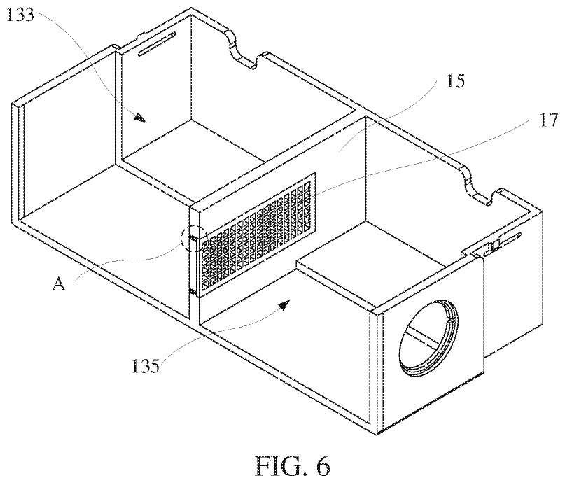

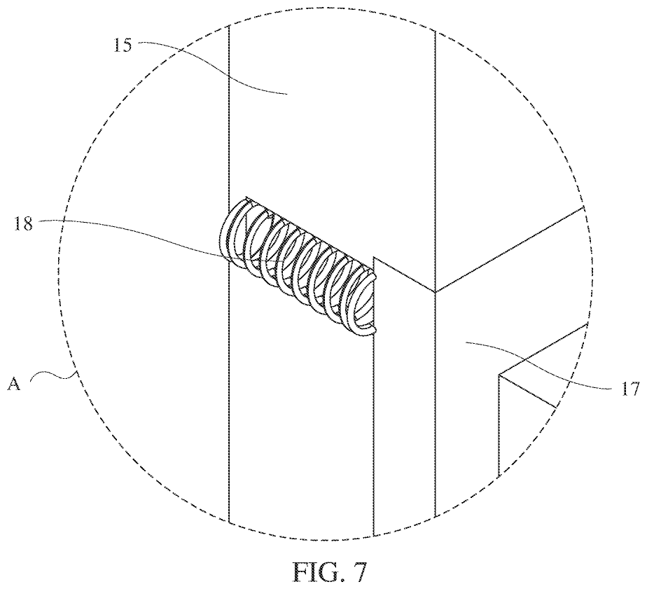

As a feasible embodiment, the housing assembly 10 further comprises an elastic piece 18, and the elastic piece 18 is arranged between the partition plate 15 and the filter piece 17. With reference to FIG. 6 and FIG. 7, the elastic piece 18 may be a spring, or an elastic steel sheet. In order to facilitate those skilled in the art to understand this solution, the following explanation will take the spring as an example, but the present application will not be limited. Two ends of the spring are respectively connected with the partition plate 15 and the filter piece 17, and the filter piece 17 is pressed with the partition plate 15 under the action of the spring in use. When in use, substances such as mucus sucked from the nasal cavity will enter the first cavity chamber 133, and then the filter piece 17 on the partition plate 15 will filter the substances such as mucus. During the filtering process, the filter piece 17 may be blocked by the substances such as mucus. When the filter piece is blocked to a certain extent, the liquid in the first cavity chamber 133 will not flow into the second cavity chamber 135 through the filter piece 17. If the blocking is not treated in time, a pressure inside the first cavity chamber 133 will gradually increase. When the pressure rises to a certain extent, the liquid inside the first cavity chamber 133 will overflow, and the negative pressure assembly 30 will be overloaded, resulting in damage to the negative pressure assembly 30. The elastic piece 18 is arranged between the filter piece 17 and the partition plate 15. If the above-mentioned blocking phenomenon occurs, with the pressure in the first cavity chamber 133 rising and reaching a preset strength, the filter piece 17 will move to the second cavity chamber 135 for a certain distance, so that a gap is formed between the filter piece and the partition plate, thereby releasing the pressure in the first cavity chamber 133, thus avoiding the overflow of liquid from the first cavity chamber 133 due to excessive pressure and damaging the negative pressure assembly 30.

As a feasible embodiment, the nasal aspirator 100 further comprises a control assembly 70, the control assembly 70 is electrically connected with the negative pressure assembly 30, an inner wall surface of the negative pressure cavity 111 is convexly provided with an accommodating step 112, the mounting groove 113 is arranged on one side of the accommodating step 112 away from the negative pressure cavity 111, and the control assembly 70 is arranged at the accommodating step 112.

It can be understood that the control assembly 70 is electrically connected with the negative pressure assembly 30 and is used to control whether the negative pressure assembly 30 is started or not, that is, whether the negative pressure environment in the negative pressure cavity 111 is generated or not, which can be controlled by buttons at least partially exposed outside the cover plate 117, further facilitating the use process of the nasal aspirator 100. On the other hand, the thin-walled accommodating step 112 is convex toward an inner wall surface of the negative pressure cavity 111, and at the same time, at least part of the mounting groove 113 is recessed on the other side. In this way, a wall surface formed in a horizontal direction of the accommodating step 112 is used to bear the control assembly 70, and a vertical wall on the side away from the mounting groove 113 is used to form a limiting space for accommodating the negative pressure assembly 30 in the negative pressure cavity 111, which further optimizes spatial arrangement in the housing assembly 10, improves space utilization ratios of the housing assembly 10 and the nasal aspirator 100, and reduces the production cost of the nasal aspirator 100.

The foregoing descriptions are merely preferred embodiments of the present invention, but are not intended to limit the patent scope of the present invention. All equivalent structure transformations made using the specification of the present invention and the accompanying drawings, or being used directly or indirectly in other related technical fields, are similarly included in the protection scope of the present invention.

What is claimed is:

1. A nasal aspirator (100), wherein the nasal aspirator (100) comprises:
a housing assembly (10), wherein a negative pressure cavity (111) and a treatment cavity (131) are arranged in the housing assembly (10) at intervals, and the treatment cavity (131) is sequentially divided into a first cavity chamber (133) and a second cavity chamber (135) by a partition plate (15), and the first cavity chamber (133) is communicated with the second cavity chamber (135);
a negative pressure assembly (30), wherein the negative pressure assembly (30) is arranged in the negative pressure cavity (111) and is formed with a suction pipeline (31) and a discharge pipeline (33) which are communicated with a side wall surface of the housing assembly (10); and
a guide assembly (50), wherein the guide assembly (50) comprises a first guide piece (51) and a second guide piece (53), the first guide piece (51) is detachably connected with the side wall surface of the housing assembly (10) to be communicated with the suction pipeline (31) and the second cavity chamber (135);
and the second guide piece (53) is fixedly connected to the side wall surface of the housing assembly (10) and is communicated with the discharge pipeline (33) and the first cavity chamber (133).

2. The nasal aspirator (100) according to claim 1, wherein the first guide piece (51) comprises:
a first guide tube (511), wherein the first guide tube (511) is made of a flexible material and arranged on the side wall surface of the housing assembly (10) to be communicated with the suction pipeline (31); and
a soft rubber dropper (513), wherein the soft rubber dropper (513) is made of a flexible material and fixedly connected to one end of the first guide tube (511) away from the housing assembly (10), one end of the soft rubber dropper (513) away from the first guide tube (511) is provided with a liquid suction port, and the liquid suction port is detachably connected to the side wall surface of the housing assembly (10) to be communicated with the first guide tube (511) and the second cavity chamber (135).

3. The nasal aspirator (100) according to claim 2, wherein the first guide piece (51) further comprises a first joint (515) and a second joint (517), the first joint (515) is arranged on one side wall surface of the housing assembly (10) close to the negative pressure cavity (111) and is communicated with the suction pipeline (31), and the second joint (517) is arranged on one side wall surface of the housing assembly (10) close to the second cavity chamber (135) and is communicated with the second cavity chamber (135); one side of the first guide tube (511) away from the soft rubber dropper (513) is communicated with the first joint (515); and the soft rubber dropper (513) is detachably connected to the second joint (517) to enable the liquid suction port to be communicated with the second joint (517).

4. The nasal aspirator (100) according to claim 2, wherein the first guide tube (511) and the soft rubber dropper (513) are integrally formed.

5. The nasal aspirator (100) according to claim 1, wherein the second guide piece (53) comprises:
a third joint (531), wherein the third joint (531) is arranged on one side wall surface of the housing assembly (10) close to the negative pressure cavity (111) and is communicated with the discharge pipeline (33);
a fourth joint (533), wherein the fourth joint (533) is arranged on one side wall surface of the housing assembly (10) close to the first cavity chamber (133) and is communicated with the first cavity chamber (133); and
a second guide tube (535), wherein the second guide tube (535) is made of a flexible material and is provided with two opposite ends to be communicated with the third joint (531) and the fourth joint (533) respectively.

6. The nasal aspirator (100) according to claim 1, wherein the housing assembly (10) comprises:
a base (11), wherein the base (11) is formed with the negative pressure cavity (111) and a mounting groove (113), and the negative pressure cavity (111) and the mounting groove (113) are arranged at intervals; and
a water tank (13), wherein the water tank (13) is detachably connected to the mounting groove (113) and enclosed with an inner wall surface of the mounting groove (113) to form the treatment cavity (131), and the partition plate (15) is arranged at the water tank (13).

7. The nasal aspirator (100) according to claim 1, wherein the base (11) comprises:
a base body (115), wherein the mounting groove (113) is arranged at the base body (115); and
a cover plate (117), wherein the cover plate (117) is detachably connected to the base body (115) and enclosed with a wall surface of the base body (115) to form the negative pressure cavity (111).

8. The nasal aspirator (100) according to claim 6, wherein a side wall surface of the water tank (13) is convexly provided with at least two clamping protrusions (137), the at least two clamping protrusions (137) are symmetrically arranged along a detachable connection direction of the water tank (13), the inner wall surface of the mounting groove (113) is concavely provided with at least two clamping grooves, and a number of the clamping grooves is set corresponding to the clamping protrusions (137) to clamp the clamping protrusions (137) when the water tank (13) is detachably connected in the mounting groove (113).

9. The nasal aspirator (100) according to claim 1, wherein the housing assembly (10) further comprises a filter piece (17), and the filter piece (17) is arranged at the partition plate (15) and is communicated with the first cavity chamber (133) and the second cavity chamber (135).

10. The nasal aspirator (100) according to claim 6, wherein the nasal aspirator (100) further comprises a control assembly (70), the control assembly (70) is electrically connected with the negative pressure assembly (30), an inner wall surface of the negative pressure cavity (111) is convexly provided with an accommodating step (112), the mounting groove (113) is arranged on one side of the accommodating step (112) away from the negative pressure cavity (111), and the control assembly (70) is arranged at the accommodating step (112).
